(19) United States
(12) Patent Application Publication
(10) Pub. No.: US 2005/0238618 A1
(43) Pub. Date: Oct. 27, 2005

(54) Low molecular weight polymers

(76) Inventor: Yujin Huang, San Diego, CA (US)

Correspondence Address: AMGEN INC., MAIL STOP 28-2-C, ONE AMGEN CENTER DRIVE, THOUSAND OAKS, CA, 91320-1799, US

(21) Appl. No.: 11/114,429

(22) Filed: Apr. 25, 2005

Related U.S. Application Data
(60) Provisional application No. 60/565,246, filed on Apr. 23, 2004.

(51) Int. Cl. A61K 31/765; A61K 9/14; A61K 9/50

(52) U.S. Cl. 424/78.37; 424/489

(57) ABSTRACT

The invention relates to a procedure for purifying low molecular weight polylactic acid polymers by use of reduced temperature liquid-liquid phase separation of the polymers in methanol, ethanol or isopropanol based solvents, compositions comprising the polymers and methods of using the same.

The Present application claims the benefit of priority to U.S. Provisional Application Ser. No. 60/656,246, filed Apr. 23, 2004.

### FIELD OF THE INVENTION

The present invention relates broadly to the field of biocompatible, biodegradable polymers. More specifically, the invention describes a method for purifying low molecular weight polymers by use of a reduced temperature liquid-liquid phase separation of a polymer solution where the solvent is comprised of methanol, ethanol and/or isopropanol. Suitable polymers useful in the methods include polylactic acid (PLA). The purified polymers of the invention are unique in their high degree of purity represented in part by having a narrower molecular weight distribution than crude polymer, thus they are particularly suitable for use in sustained release formulations or biocompatible polymers.

### BACKGROUND

Diagnostic agents and drugs, whether protein or small molecule, each have a defined half life in the body of a patient. Oftentimes the effect of the agent or drug can be maximized by extending its half life. One method is to encapsulate the agent or drug in a material that is biocompatible with the subject to which it is administered, where the material slowly breaks down or dissolves such that the release of the agent or drug is over a sustained period longer than the half life of the agent or drug alone.

It has been shown that one can encapsulate a biologically active or pharmaceutically active agent within a biocompatible, biodegradable wall forming material such as a polymer, to provide sustained or delayed release. In these methods the agent or drug is typically dissolved, dispersed or emulsified, using stirrers, agitators, or other dynamic mixing techniques, in one or more solvents containing the wall forming material. The solvent is then removed resulting in the formation of microparticles encapsulating the agent or drug. These microparticles can then be administered to a patient.

Biodegradable polymers have been extensively used in controlled drug delivery. They have the advantage of not requiring surgical removal after they serve their intended purposes due to the fact that they are degraded either enzymatically or chemically, e.g., hydrolysis. Polyesters such as poly(lactic acid) (PLA), poly(glycolic acid) (PGA), and poly(lactic-co-glycolic acid) (PLGA) have been extensively studied for a wide variety of pharmaceutical and biomedical applications. PGA, PLA, and especially their copolymers PLGA are the most commonly used family of polymers. Each of these polymers exhibits the desired characteristics of biocompatibility and are biodegradable when injected into a patient, and therefore they have achieved wide acceptance as pharmaceutical components, and particularly for sustained release formulations. Chaubal, Drug Delivery Technology, 2002, 2:34-36 and Anderson et al., Adv. Drug Deliv. Rev., 1997, 28:5-24.

A drug encapsulated in a PLA microparticle is released either from the diffusional effects of the aqueous environment or the degradation of the polymer, mentioned above. One variable that affects the in vitro and in vivo drug release rate of a microparticle made from a polymer, is its molecular weight. In particular, the molecular weight of a polymer influences the biodegradation rate. For a diffusional mechanism of active agent release, the polymer should remain intact until all of the active agent is released from the microparticles, and then degrade. The active agent can also be released from the microparticles as the polymeric matrix material bioerodes.

It has also been shown that lower molecular weight polymers tend to release the active agent in a more rapid manner than high molecular weight polymers (Asano et al., Biomaterials, 1989, vol. 10:569). Thus, by selecting lower molecular weight polymers, a formulation can be made in which the resulting microparticles exhibit accelerated release. This is desirable when the active agent needs to be delivered in a shorter period or at higher concentrations. However, there are no consistent techniques available to prepare relatively pure low molecular weight polymers that have low polydispersity (Asano et al., Biomaterials, 1989, vol. 10:569; Hyon et al., Biomaterials, 1997, vol. 18:1503).

Another factor in the manufacture of polymers for use in sustained release formulations is the polydispersity of the polymer mixture. There is no consistent method that results in the production or purification of smaller molecular weight polymers having relatively low polydispersity. Accordingly, there is a need in the art for an improved method for preparing polymer microparticles for sustained release pharmaceutical preparations (e.g., such as microparticles, rods, films, and the like) where the method results in low molecular weight polymers having low polydispersity.

### SUMMARY OF THE INVENTION

The present invention teaches a method for purifying low molecular weight polymers by use of a reduced temperature liquid-liquid phase separation of a polymer solution where the solvent is comprised of mixture of methanol, ethanol and/or isopropanol. In specific embodiments, the solvent is methanol, ethanol or isopropanol.

The present invention relates to a method of producing low molecular weight and low polydisperse polymers. More specifically, the invention provides a method of purifying low molecular weight polymers using reduced temperature phase separation of polymers in a single phase solvent, where the solvent is selected from the group consisting of methanol, ethanol and isopropanol. More particularly, the method comprises the steps of mixing the crude polymer with methanol, ethanol or isopropanol until the polymer is dissolved, reducing the temperature of the solution until two-layers form, separating the upper layer liquid from the lower layer and isolating the polymer. It is also contemplated that the solvent can be a mixture of methanol, ethanol or isopropanol with each other or other liquids, so long as the solution used acts as a solvent for the polymer (e.g., PLA) of the invention and is capable of phase separation as described herein.

In one embodiment, the solvent is primarily methanol, ethanol, or isopropanol. It is contemplated that the primary solvent can be mixed with other solvents. For example, methanol and ethanol could be mixed and used according to the methods of the invention or methanol could be mixed with another solvent such as methylene chloride where the solvent mixture still allows for purification of low molecular weight, less polydisperse polymers.

In certain embodiments, the choice of polymer is poly(lactic acid), known as PLA. In a particular embodiment, upon purification using the methods of the invention, the low molecular weight polymer has a P value, measured by dividing Mw by Mn, that is less than 1.6. Accordingly, it is an embodiment of the invention that the polymers purified by the methods of the invention have a narrower molecular weight distribution than traditionally manufactured polymers.

The polymer can be manipulated to form microparticles, rods, films and the like, suitable for injection into a patient in need thereof. Accordingly, the invention also relates to pharmaceutically acceptable formulations of the polymers purified as described herein and methods of using the same.

### DETAILED DESCRIPTION

The present invention relates to a procedure for purifying low molecular weight polymers by use of a reduced temperature liquid-liquid phase separation of the polymer in methanol, ethanol or isopropanol, compositions comprising the polymers and methods of using the same. In one embodiment, the choice of polymer is poly (lactic acid), known as PLA. The resulting polymers have a narrower molecular weight range and are markedly purer than those derived using currently available methods. Thus the invention also relates to the novel composition of purified PLA polymers, and their use in standard pharmaceutical compositions such as microparticles. It is contemplated that the purified low molecular weight, low polydisperse polymers can be used to generate microparticles that encapsulate agents and/or drugs suitable for injection into a patient in need thereof. It is further contemplated that the purified low molecular weight, low polydisperse polymers of the invention, when made into microparticles, will provide for a sustained release of the agent and/or drug.

As used herein, it is understood that the phrase “low molecular weight” is intended to indicate a range of molecular weights where the average molecular weight is less than 5,000 Daltons. In alternative embodiments, low molecular weight indicates an average that is less than 3,000 Daltons. When this phrase is used in conjunction with a polymer, it is understood that the preferred embodiment is poly (lactic acid), known as PLA. In one embodiment, the PLA polymer is substantially comprised of moieties containing lactic acid ester.

In one embodiment, the purification method of the invention results in a low molecular weight polymer that has lower polydispersity than previously disclosed polymer purification steps. In this embodiment, it is contemplated that the polymer has a P value measured by dividing Mw by Mn that is less than 1.6. More preferably the P value is less than 1.55, more preferably the P value is below 1.5, more preferably the P value is below 1.45, more preferably the P value is below 1.4, and most preferably is less than 1.3. In addition, the methods of the invention result in a polymer form that is a free flowing fine white powder. In yet another embodiment, the fractionated polymers of the invention when dried are very white, having the appearance of being snow white colored.

In another embodiment, it is contemplated that the polymer produced according to the invention has a weight average molecular weight of 800 to 10,000, 800 to 5,000, 800 to 4,000, 800 to 3,000, 800 to 2,000, 800 to 1,500, 800 to 1,200, or 1,000 to 2,000, or 1,000 to 1,500, or 1,000 to 1,200.

While the representative examples utilize methanol, ethanol or isopropanol, one of skill in the art would readily understand that mixtures of these three alcohols, or solvents containing less than 100% of any one of the three may also be used. For example, ethanol could be diluted with another solvent such as isopropanol to obtain a 90% ethanol, 10% isopropanol solvent that would also work according to the teachings of the present invention. It is also contemplated that solvents other than the three primary solvents could be added to create a solvent mixture, for example, methylene chloride. One of skill in the art will be able to determine the appropriate limits to the dilution of the primary solvents, i.e., methanol, ethanol and isopropanol, with other solvents such that the resulting mixed solvent can be used according to the teachings herein.

In further embodiments, it will be understood by one of skill in the art that the initial dissolution of the unpurified polymer in a solvent of the invention will depend on the solubility of the polymer. Thus, it is contemplated that the solvent-polymer mixture may need to be warmed above room temperature for the polymer to dissolve. The subsequent phase separation step, which is facilitated by a reduction in temperature may occur at a lower temperature, for example, at or near room temperature. Accordingly, the starting temperature where the polymer is dissolved in the solvent may be around 60° C. and the phase separation may be at or around room temperature (e.g., 18° C. to 28° C.). In another example, the starting temperature where the polymer is dissolved in the solvent may be room temperature and the phase separation occurs at around 10° C. or lower.

One of skill in the art, using the teachings herein, will be able to readily determine the appropriate dissolving temperature and the phase separation temperature using routine experimentation. Thus, in further embodiments, the starting temperature may be about 10 degrees centigrade above the phase separation temperature. In one example, if the polymer is dissolved at room temperature, then it is contemplated that the phase separation temperature is at least about 10° C. cooler than the dissolving temperature, e.g., about 10° C., or alternatively about 5° C., about 0° C., about −5° C., about −10° C., about −15° C., about −20° C., about −30° C., about −40° C., about −50° C., about −60° C. or lower, such that phase separation occurs. Based on the teachings herein, one of skill in the art will be able to readily determine the best temperatures for both dissolving the polymer and triggering the phase separation using routine experimentation.

As used herein, the term ‘about’ is meant to reflect a variability of up to 20% of the enumerated value, whether it is temperature as described immediately above, or is for another value.

An optimal solubility needs to be determined experimentally, since too high a solubility will negate a liquid-liquid phase separation while too low a solubility will be impractical. It is convenient to use the USP definitions of solubility where slightly soluble is one part polymer to 100-1000 parts solvent, and increasing solubility to 30-100 parts solvent, further increasing solubility to 10-30 parts solvent, even further increasing solubility to 1-10 parts solvent and very soluble is one part polymer to less than one part solvent.

As used herein, the term “microparticles” refers to particles having a volume median particle size of between about 1 and 1000 microns. Furthermore, the term “non-solvent” refers to a material which does not substantially dissolve a substance and a “solvent” is understood to refer to a liquid that dissolves the polymer of the invention. As used herein, a “sustained release” of an agent and/or drug is a release from the composition of the invention which occurs over a period which is longer than that period during which an agent and/or drug would be available following direct administration.

It is contemplated that sustained release of an agent, encapsulated in microparticles made from the polymers purified according to the invention, occurs over a period of greater than one day. Sustained release can be a continuous or a discontinuous release, with relatively constant or varying rates of release. The continuity of release and level of release can be affected by the type of polymer composition used (e.g., monomer ratios, molecular weight, block composition, and varying combinations of polymers), protein loading, and/or selection of excipients to produce the desired effect.

Suitable biocompatible polymers that can be purified according to the methods described herein, can be either biodegradable or non-biodegradable polymers or blends or copolymers thereof. A polymer is biocompatible if it and any degradation products are non-toxic to the recipient. More particularly, non-toxic is intended to encompass no significant deleterious or untoward effects on the recipient's body in the normal course of use of the polymers of the invention, such as a significant immunological reaction to the injection due to the polymer.

One suitable biocompatible, biodegradable polymer that can be purified and used according to the present invention includes, for example, polylactic acids (PLA). Other polymers known in the art that may be suitable for purification according to methods similar to those described herein include polyglycolides (PGA), polylactide-co-glycolides (PLGA), poly(lactic acid)s, poly(glycolic acid)s, polycarbonates, polyesteramides, polyanydrides, poly(amino acids), polyorthoesters, poly(dioxanone)s, poly(alkylene alkylate)s, copolymers or polyethylene glycol and polyorthoester, biodegradable polyurethane, blends thereof, and copolymers thereof.

The term “biodegradable” is understood to mean the composition will degrade or erode in vivo to form smaller chemical species. Degradation can result, for example, by enzymatic, hydrolytic or other chemical mechanisms, and/or physical processes. The release of an encapsulated biologically active agent from a biodegradable PLA formulation of the invention is by a combination of diffusion and degradation, i.e., enzymatic or hydrolytic, of the polymer composition.

The term “biologically active agent,” as used herein, is an agent, or its pharmaceutically acceptable salt, which when released in vivo, possesses the desired biological activity, for example therapeutic, diagnostic and/or prophylactic properties in vivo. A sustained release composition of the invention can contain from about 0.01% (w/w) to about 90% (w/w) of active agent (dry weight of composition). The amount of agent can vary depending upon the desired effect of the agent, the planned release levels, and the time span over which the agent is to be released. Examples of suitable biologically active agents include proteins, peptides, muteins and active fragments thereof and also small molecules, described more fully below.

As used herein, the terms “protein” and “peptide” are understood to include polymers of amino acids linked by amide bonds. Typically, a peptide will be composed of less than about 50 amino acids, more typically less than about 30 amino acid residues and even more typically, less than about 20 amino acid residues. Whereas a protein will typically be composed of more than 50 amino acids and will have structure and biological activity. The protein's biological activity can be enzymatic or it may be a binding activity that confers conformation changes. These terms are further intended to encompass analogues and derivatives that mimic the chemical structure of the components of the protein or peptides. Examples of analogues include peptides or proteins containing one or more non-natural amino acids. Examples derivatives include peptides or proteins containing amino acid side chain(s), peptide backbone, and/or amino- or carboxy-terminus that have been derivatized.

Peptides suitable for formulation according to the invention include but are not limited to enfuvirtide (sold by Trimeris and Roche as Fuzeon©), Angiotensin, Amylin, ACTH, renin substrate, Cecropin A-Melittin amide, Cecropin B, Magainin 1, Renin Inhibitor Peptide, Bombesin, Osteocalcin, Bradykinin, B 1 Inhibitor Peptide, Kallidin, Calcitonin, Cholecystokinin, Corticotropin Releasing Factor, Dynorphin A, Endomorphin, Sarafotoxin, Enkephalin, Exendin, Fibrinopeptide, Galanin, Gastrin, Gastrin Releasing Peptide, Glucagon-Like Peptide, Growth Hormone Releasing Factor, OVA Peptide, Luteinizing Hormone-Releasing Hormone, Atrial Natriuretic Peptide, Melanin Concentrating Hormone, Brain Natriuretic Peptide, Vasonatrin, Neurokinin, Neuromedin, Neuropeptide Y, Neurotensin, Orexin, Oxytocin, Vasopressin, Parathyroid Hormone Peptide, Prolactin Releasing Peptide, Somatostatin, Somatostatin Tumor Inhibiting Analog, Thyrotropin Releasing Hormone, and variants and derivatives thereof (see also, Latham, (1999) Nat. Biotech., 17:755).

Examples of suitable proteins, muteins and active fragments thereof, include but are not limited to immunoglobulins, antibodies, cytokines (e.g., lymphokines, monokines, chemokines), interleukins, interferons (beta-IFN, alpha-IFN and gamma-IFN), erythropoietin, nucleases, tumor necrosis factor, colony stimulating factors, insulin, enzymes (e.g. superoxide dismutase, tissue plasminogen activator), tumor suppressors, blood proteins, hormones and hormone analogs (e.g., growth hormone, adrenocorticotropic hormone and luteinizing hormone releasing hormone (LHRH)), vaccines (e.g., tumoral, bacterial and viral antigens), antigens, blood coagulation factors; growth factors; peptides such as protein inhibitors, protein antagonists, and protein agonists; nucleic acids, such as antisense molecules; oligonucleotides; and ribozymes.

Small molecular weight agents suitable for use in the invention include, antitumor agents such as bleomycin hydrochloride, carboplatin, methotrexate and adriamycin; antibiotics such as gentamicin, tetracycline hydrochloride and ampicillin; antipyretic, analgesic and anti-inflammatory agents; methylephedrine hydrochloride, noscapine hydrochloride and codeine phosphate; sedatives such as chlorpromazine hydrochloride, prochlorperazine hydrochloride and atropine sulfate; muscle relaxants such as tubocurarine chloride; antiepileptics such as sodium phenyloin and ethosuximide; antiulcer agents such as metoclopramide; antidepressants such as clomipramine; antiallergic agents such as diphenhydramine; cardiotonics such as theophillol; antiarrhythmic agents such as propranolol hydrochloride; vasodilators such as diltiazem hydrochloride and bamethan sulfate; hypotensive diuretics such as pentolinium and ecarazine hydrochloride; antidiuretic agents such as metformin; anticoagulants such as sodium citrate and sodium heparin; hemostatic agents such as thrombin, menadione sodium bisulfite and acetomenaphthone; antituberculous agents such as isoniazide and ethanbutol; hormones such as prednisolone sodium phosphate and methimazole; antipsychotic agents such as risperidone; and narcotic antagonists such as nalorphine hydrochloride.

“Stabilizing agent”, as that term is used herein, is any agent which binds or interacts in a covalent or non-covalent manner or is included with the biologically active agent. Stabilizing agents suitable for use in the invention are described in U.S. Pat. Nos. 5,716,644, 5,674,534, 5,654,010, 5,667,808, and 5,711,968.

Further, excipients can be added to maintain the potency of the biologically active agent over the duration of release and modify polymer degradation. The excipients can be added to the dispersed system which is then atomized or can be added to the mixture which is subjected to fragmenting either before or after fragmentation of the dried substance to achieve particles of biologically active agent. Suitable excipients include, for example, carbohydrates, amino acids, fatty acids, surfactants, and bulking agents, and are known to those skilled in the art. An acidic or a basic excipient is also suitable. The amount of excipient used is based on its ratio to the biologically active agent, on a weight basis. For amino acids, fatty acids and carbohydrates, such as sucrose, trehalose, lactose, mannitol, dextran and heparin, the ratio of carbohydrate to biologically active agent, is typically between about 1:10 and about 20:1. For surfactants the ratio of surfactant to biologically active agent is typically between about 1:1000 and about 2:1. Bulking agents typically comprise inert materials. Suitable bulking agents are known to those skilled in the art.

The excipient can also be a metal cation component which is separately dispersed within the polymer matrix. This metal cation component acts to modulate the release of the biologically active agent and is not complexed with the biologically active agent. The metal cation component can optionally contain the same species of metal cation, as is contained in the metal cation stabilized biologically active agent, if present, and/or can contain one or more different species of metal cation. The metal cation component acts to modulate the release of the biologically active agent from the polymer matrix of the sustained release composition and can enhance the stability of the biologically active agent in the composition. Examples of metal cation components suitable to modulate release include or contain, for example, Na, K, Mg, Zn, and Ca. The optimum ratio of cation to polymer depends upon the polymer and the metal cation component utilized and will be readily determined by one of skill in the art. A polymer matrix containing a dispersed metal cation component to modulate the release of a biologically active agent from the polymer matrix is further described in U.S. Pat. No. 5,656,297.

In yet another embodiment, at least one pore forming agent, such as a water soluble salt can be included in a sustained release composition to modify the microstructure, for example, as taught in U.S. Pat. No. 6,531,154. The proportion of pore forming agent added to the suspension comprising submicron particles of biologically active agent dispersed in a solution comprising at least one biocompatible polymer and at least one polymer solvent, is between about 1% (w/w) to about 30% (w/w).

A number of methods are known by which polymer/active agent matrices can be formed. In many of these processes, the material to be encapsulated is dispersed in a solvent containing a wall forming material. At a single stage of the process, solvent is removed from the microparticles and thereafter the microparticle product is obtained. For example, methods for forming a composition for the sustained release of biologically active agent are described in U.S. Pat. No. 5,019,400 and U.S. Pat. No. 5,922,253. While the most suitable polymer for the methods of the invention is PLA, one of skill in the art will recognize that the PLA polymer purified by the methods described herein could be mixed with other polymers in the preparation of a sustained release formulation. These blends of polymers can be used in the formation of matrices suitable for drug delivery. It will also be understood that these blends will often have differing properties from pure forms of PLA molecules of the invention used accordingly.

Means suitable for freezing droplets include directing the droplets into or near a liquified gas, such as liquid argon or liquid nitrogen to form frozen microdroplets which are then separated from the liquid gas. The frozen microdroplets are then exposed to a liquid or solid non-solvent, such as ethanol, hexane, ethanol mixed with hexane, heptane, ethanol mixed with heptane, pentane or oil. The solvent in the frozen microdroplets is extracted as a solid and/or liquid into the non-solvent to form a polymer/active agent matrix comprising a biocompatible polymer and a biologically active agent. Mixing methanol, ethanol or isopropanol with other non-solvents, such as hexane, heptane or pentane, can increase the rate of solvent extraction, above that achieved by methanol, ethanol or isopropanol alone, from certain polymers.

A wide range of sizes of sustained release compositions can be made by varying the droplet size, for example, by changing the ultrasonic nozzle diameter. If the sustained release composition is in the form of microparticles, and very large microparticles are desired, the microparticles can be extruded, for example, through a syringe directly into the cold liquid. Increasing the viscosity of the polymer solution can also increase microparticle size. The size of the microparticles which can be produced by this process ranges, for example, from greater than about 1000 to about 1 micrometers in diameter.

Yet another method of forming a sustained release composition, from a suspension comprising a biocompatible polymer and a biologically active agent, includes film casting, such as in a mold, to form a film or a shape. For instance, after putting the suspension into a mold, the polymer solvent is then removed by means known in the art, or the temperature of the polymer suspension is reduced, until a film or shape, with a consistent dry weight, is obtained.

A further example of a conventional microencapsulation process and microparticles produced thereby is disclosed in U.S. Pat. No. 3,737,337, wherein a solution of a wall or shell forming polymeric material in a solvent is prepared. The solvent is only partially miscible in water. A solid or core material is dissolved or dispersed in the polymer-containing mixture and, thereafter, the core material-containing mixture is dispersed in an aqueous liquid that is immiscible in the organic solvent in order to remove solvent from the microparticles.

Another example of a process in which solvent is removed from microparticles containing a substance is disclosed in U.S. Pat. No. 3,523,906. In this process a material to be encapsulated is emulsified in a solution of a polymeric material in a solvent that is immiscible in water and then the emulsion is emulsified in an aqueous solution containing a hydrophilic colloid. Solvent removal from the microparticles is then accomplished by evaporation and the product is obtained.

In still another process as shown in U.S. Pat. No. 3,691,090, organic solvent is evaporated from a dispersion of microparticles in an aqueous medium, preferably under reduced pressure. Similarly, the disclosure of U.S. Pat. No. 3,891,570 shows a method in which solvent from a dispersion of microparticles in a polyhydric alcohol medium is evaporated from the microparticles by the application of heat or by subjecting the microparticles to reduced pressure. Another example of a solvent removal process is shown in U.S. Pat. No. 3,960,757.

Tice et al., in U.S. Pat. No. 4,389,330 describe the preparation of microparticles containing an active agent by a method comprising: (a) dissolving or dispersing an active agent in a solvent and dissolving a wall forming material in that solvent; (b) dispersing the solvent containing the active agent and wall forming material in a continuous-phase processing medium; (c) evaporating a portion of the solvent from the dispersion of step (b), thereby forming microparticles containing the active agent in the suspension; and (d) extracting the remainder of the solvent from the microparticles.

Without being bound by a particular theory it is believed that the release of the biologically active agent can occur by two different mechanisms. First, the biologically active agent can be released by diffusion through aqueous filled channels generated in the polymer matrix, such as by the dissolution of the biologically active agent, or by voids created by the removal of the polymer solvent during the preparation of the sustained release composition. A second mechanism is the release of the biologically active agent, due to degradation of the polymer. The rate of degradation can be controlled by changing polymer properties that influence the rate of hydration of the polymer. These properties include, for instance, the ratio of lactide to glycolide, comprising a polymer; the use of the L-isomer of a monomer instead of a racemic mixture; and the molecular weight of the polymer. These properties can affect hydrophilicity and crystallinity, which control the rate of hydration of the polymer.

The polymers of the invention and pharmaceutically acceptable variants thereof can be administered in vivo, for example, to a human or to an animal, by injection, implantation (e.g., subcutaneously, intramuscularly, intraperitoneally, intracranially, and intradermally), administration to mucosal membranes (e.g., intranasally, intrapulmonary, buccally or by means of a suppository), or in situ delivery (e.g., by enema or aerosol spray) to provide the desired dosage of biologically active agent based on the known parameters for treatment with the particular agent of the various medical conditions. As used herein, a “therapeutically effective amount”, “prophylactically effective amount” or “diagnostically effective amount” is the amount of the biologically active agent or of the sustained release composition of biologically active agent needed to elicit the desired biological, prophylactic or diagnostic response following administration.

The following examples are understood to be representative working examples and are not intended to limit the full scope of the claimed invention. The number average and weight average molecular weights (Mn, Mw) of the PLA polymers described below were determined by end group titration and gel permeation chromatography (GPC; universal calibration).

### EXAMPLES

Synthesis of Low Molecular Weight Polylactic Acid (PLA) Polymers

The synthesis of PLA polymers through polycondensation of lactic acid monomer was performed in the absence of any catalyst by distilling out water from 85 weight percent aqueous solution of lactic acid at high temperatures and reduced pressure. For example, 412 grams of aqueous lactic acid solution was charged into a 500 ml three-necked flask fitted with a stir bar inside, a water-cooling condenser through a distillation head with a thermometer, a needle inlet (connected with a gas bubbler and inserted into a rubber septum to pass through dry nitrogen gas). Under atmospheric pressure, the rate of nitrogen was around 280 bubbles per minute.

The condenser was connected to an adaptor which was linked to a gas bubbler and receiving flask. The upper part of flask was wrapped with glass fiber. The flask was immersed into oil bath until the level of liquid was equal to oil level. The variable transformer was always set at 70 and 140 v. The stirring position of hot plate was at 8. The flask was heated on an oil bath from room temperature to 140° C. during a 50 minute time period. When water started to condense, the temperature was gradually raised to 160° C. over the course of around two hours. Then the adaptor was connected to a Buchi Rotavapor pump system instead of a gas bubbler and the receiving flask was cooled by a dry ice bath. The pressure was reduced from atmosphere to 400 mbar and the oil bath temperature was gradually increased to 170° C. during a period of 40 minutes. The rate of nitrogen was decreased to 2-10 bubbles per minute. The system pressure was further reduced to 100 mbar and the oil bath temperature was gradually increased to 188° C. for around 55 minutes. Reducing pressure must be gradual in order to prevent bumping of distillation and the distillation head temperature was not above 120° C.

The reaction was stirred under these conditions for 1,3 5 and 7 hr preparing PLA of Mn approximately 700, 1000, 1500 and 2000 respectively. The oil bath was removed and the flask was flooded with nitrogen and cooled to room temperature. The flask was stored in the freezer (−40° C.) for next day purification.

TABLE 1
Conditions for direct condensation of DL-lactic acid

| | Scale | Vacuum, | Time (hr) at | Appearance after |
|---|---|---|---|---|
| Mn Range | (monomer, g) | mbar | 188 C. | purification |
| 2K | 419 | 100 | 7 | White solid |
| 1.5K | 418 | 100 | 5 | White solid |
| 1K | 412 | 100 | 3 | White solid |

Purification of low PLA Polymers:
Mn Approximately 700
Polymers Prepared at 1 hr at 188° C. Under 100 mbar Vacuum
Method B:

A prior art method was used to prepare polymers. To a flask was added 220 ml dichloromethane and the mixture was heated on an oil bath at 55° C. with gentle refluxing until the polymer was completely dissolved (around 2-3 hr). The solution was then poured into 400 ml deionized (DI) water in a 1 liter beaker and the mixture was stirred for 0.5 hour. Extra dichloromethane (180 ml) was added to help separate layers in the funnel. The organic layer (around 450 ml) was separated in separatory funnel. Dichloromethane was removed by rotavapor under reduced pressure. The gel-like polymer was further dried under vacuum for three days. 153 g of gel PLA (Mn; 670) was obtained.

Mn Approximately 1000

Polymers Prepared at 3 hr at 188° C. Under 100 mbar Vacuum

Method E:

To the flask was added 220 ml dichloromethane and the mixture was heated on an oil bath at 55° C. with gentle reflux until the polymer was completely dissolved (around 2-3 hr). The solution was then poured into 400 ml DI water in a 1-liter beaker and the mixture was stirred for 0.5 hour. Extra dichloromethane (180 ml) was added. The organic layer (around 450 ml) was separated in a separator funnel. Dichloromethane was removed using a Rotavapor under reduced pressure. The gel-like polymer was further dried by Rotavapor less than 2 mm Hg vacuum at 35° C. water bath temperature for 3 hr.

The crude polymer was transferred into a 500 ml plastic container and mixed in 320 ml ethanol at room temperature and stored at −40° C. for 4 hr. A two-layer mixture formed. The upper layer liquid was quickly removed. Another 200 ml of ethanol was mixed with polymer in the remaining bottom layer at room temperature, and then cooled at −78° C. A white solid formed and was isolated by decanting the solution. The polymer was washed with 200 ml of pentane at −78° C. and lyophilized for 5 days. 136.4 g white solid PLA (Mn: 1042) was obtained. The upper layer solution and washing liquid were combined. The ethanol solvent of the upper layer was removed by rotavapor under reduced pressure and the residue was dried over vacuum for 5 days. 27.1 g of gel like PLA (Mn: 679) was obtained.

Mn Approximately 1500

Polymers Prepared at 5 hr at 188° C. Under 100 mbar Vacuum

Method E:

To the flask was added 220 ml dichloromethane and the mixture was heated on an oil bath at 55° C. with gentle reflux until the polymer was completely dissolved (around 2-3 hr). The solution was then poured into 400 ml of 60° C. DI water in a liter beaker and the mixture was stirred for 0.5 hour. Extra dichloromethane (130 ml) was added. The organic layer was separated in a separatory funnel. During a 2.5 hr period, dichloromethane of PLA solution (around 440 ml) was added dropwise by a syringe pump with mechanical stirring to 3400 ml of ethanol contained in 4 l of beaker, which was cooled by a dry ice/acetone bath. A white solid precipitated. After completing the addition, the mixture stood for 1-2 hr and most of the solution was poured off. The polymer was divided into two 500 ml containers and cooled again at −78° C. A solid formed and solution was removed.

To each container was mixed 200 ml of ethanol at room temperature. The mixture stored at −40° C. for 4 hr. Two cloudy layers formed. The upper layer was removed (bottom layer was slightly solidified at this temperature). The bottom layer was cooled at −78° C. to remove more of the remaining ethanol and washed with 2×200 ml pentane at 78° C.

The polymer was dried under vacuum for ten days to give a white solid (119 g, Mn: 1589 PLA).

Mn Approximately 2000

Polymers Prepared at 7 hr at 188° C. Under 100 mbar Vacuum

Method E:

This procedure was similar to the procedure described above, (Mn Approximately 1500) and yielded a white solid (112 g, Mn: Approximately 2000). Results are shown in Table 2.

TABLE 2
Additional Experiments of Low Molecular Weight
Parameters of PLA Polymers Purified by
Different Methods

| | | | GPC | | |
|---|---|---|---|---|---|
| Method | Purification methods | Titration Mn | Mn | Mw | Mw/Mn |
| A | Crude PLA product | 839 | 1061 | 1829 | 1.722 |
| B | PLA was dissolved in | 1251 | 1131 | 1835 | 1.622 |
| | dichloromethane, washed with hot | | | | |
| | water (60° C.), then water and | | | | |
| | precipitated from cold ethanol (dry | | | | |
| | ice-acetone). | | | | |
| C | PLA was dissolved in | 1068 | 1249 | 2020 | 1.617 |
| | dichloromethane, washed with water | | | | |
| | and precipitated from cold ethanol | | | | |
| | (dry ice-acetone) | | | | |
| D | PLA was dissolved in | 1068 | 1157 | 1999 | 1.727 |
| | dichloromethane and precipitated from | | | | |
| | cold ethanol (dry ice-acetone) | | | | |
| E | From method C, the PLA was mixed | 1556 | 1614 | 2254 | 1.396 |
| | with ethanol at room temperature, | | | | |
| | stored at −40° C.; two layers formed | | | | |
| | and upper layer was discarded; bottom | | | | |
| | layer collected | | | | |

Low Molecular Weight PLA Purified by Phase Separation of Polymer Solutions

Methanol and ethanol have been reported as non-solvents for PLA (Mn>800) in the literature. The following example demonstrates that low molecular weight (MW) PLA (Mn<2000) can dissolve in MeOH (methanol) and EtOH (ethanol) at room temperature and elevated temperature (approximately 40-50° C.) depending on the MW of polymer. Also, isopropanol (IPA) is shown to dissolve PLA at room temperature and up to 50° C. depending on the MW of the PLA starting material. A comparison of the solubility of a common PLA material showed that the general order of PLA solubility in these solvents is MeOH>EtOH>IPA. In other words, the PLA polymers tested were more soluble in methanol, followed by ethanol and were least soluble in isopropanol.

Liquid-liquid phase separation of polymer solution by temperature reduction was observed in alcohol solvent systems, such as MeOH, IPA and MeOH-glycerol (Table 3). The critical temperature for such phase separation of PLA solution in a single solvent system was not limited to below room temperature; it was also brought about at room temperature, such as the phase separation in PLA-IPA system at room temperature.

The molecular weight distribution of low MW PLA can be further narrowed after phase separation in either the top (lower MW) or bottom phase (higher MW), via continued purification by the phase separation method in these alcohol solvent systems. The results analyzed by GPC coupled on-line dual detectors (RI and viscometry) are listed in Table 3.

The phase separation of PLA solution by temperature reduction was not observed in solvents with strong solubilizing power such as dichloromethane (DCM), acetone, acetonitrile (ACN) and ethyl acetate. Although polymer precipitation can be brought about by addition of non-solvents into these solvents to reduce their solubilizing power, the polymer fractionation in these systems was poor due to almost complete precipitation of polymer in the bottom phase (Table 3).

TABLE 3
Molecular weight and polydispersity (Mw/Mn) of low MW PLA polymers
obtained from a second phase separation fractionation

| | Top Phase | | | Bottom Phase | | |
|---|---|---|---|---|---|---|
| Sample/Solvent | Mn | Mw | Mw/Mn | Mn | Mw | Mw/Mn |
| Single solvent/ | | | | | | |
| temperature reduction | | | | | | |
| PLA/MeOH | 1030 | 1381 | 1.34 | 1618 | 2256 | 1.39 |
| PLA/EtOH | 1125 | 1352 | 1.27 | 1751 | 2314 | 1.32 |
| PLA/IPA | 1125 | 1352 | 1.27 | 1962 | 2577 | 1.31 |
| Binary solvent/Room | | | | | | |
| temperature | | | | | | |

| | | | | |
|---|---|---|---|---|
| PLA/DCM-Hexane | Note 5 | 1023 | 1657 | 1.62 |
| PLA/Ethyl acetate- | Note 5 | 1017 | 1604 | 1.58 |
| Hexane | | | | |

| | Mn | Mw | Mw/Mn |
|---|---|---|---|
| Crude PLA | 790 | 1394 | 1.77 |

Notes:
1) Molecular weights were determined by GPC using Universal Calibration.
2) The PLA in MeOH system was phase separated at −20° C.
3) The PLA in EtOH system was phase separated at 4° C.
4) The PLA in IPA system was phase separated at RT.
5) The PLA in binary solvent/non-solvent (1:1) mixtures, e.g. DCM/hexane and ethyl acetate/hexane was separated at RT and little PLA was found in the top layer phase, i.e., PLA almost completely precipitated in the bottom phase.

While the present invention has been described in terms of preferred embodiments, it is understood that variations and modifications will occur to those skilled in the art. Therefore, it is intended that the appended claims cover all such equivalent variations which come within the scope of the claims.

1. A method of fractionating low molecular weight polylactic acid (PLA) polymers comprising the steps of:
A) dissolving the PLA polymer in a solvent, wherein the solvent is a mixture that contains methanol, ethanol or isopropanol;
B) cooling the solution of step A to induce liquid-liquid phase separation; and
C) separating the upper and lower layers of step B.

2. The method of claim 1, further comprising the steps of:
D) adding solvent to the separated top and/or bottom layer;
E) allowing the mixtures of step D to form a solid, and
F) isolating the solids from each layers.

3. The method of claim 1, wherein the solvent is comprised of a solvent selected from the group consisting of methanol, ethanol or isopropanol.

4. The method of claim 1, wherein the solvent is comprised of methanol.

5. The method of claim 1, wherein the solvent is comprised of ethanol.

6. The method of claim 1, wherein the solvent is comprised of isopropanol.

7. The method of claims 3, 4, 5 or 6, further comprising the steps of repeating steps A-C.

8. A solid polymer isolated from a layer of any of claims 1 or 2.

9. A solid polymer made by the method of claim 7.

10. The polymer of claim 9, wherein the polydispersity is less than 1.6.

11. The polymer of claim 10, wherein the polydispersity is less than 1.3.

12. The polymer of claim 8, wherein the number average molecular weight of the polymer is between 800 and 2,500.

13. A pharmaceutically acceptable composition comprising the purified polymer of claim 2.

14. A solid PLA polymer, wherein the polydispersity is less than 1.6.

15. The polymer of claim 14, wherein the polydispersity is less than 1.3.

16. A method of treating a condition comprising administering an effective amount of a pharmaceutical composition comprising a microparticle, wherein the microparticle is made from the PLA polymer of claim 14.
